(19) United States
(12) Patent Application Publication
(10) Pub. No.: US 2006/0217290 A1
(43) Pub. Date: Sep. 28, 2006

(54) Insulin analogs having protracted time action

(76) Inventors: Wayne David Kohn, Indianapolis, IN (US); Lianshan Zhang, Carmel, IN (US); Richard Dennis Dimarchi, Carmel, IN (US)

Correspondence Address: ELI LILLY & COMPANY, PATENT DIVISION, P.O. BOX 6288, INDIANAPOLIS, IN, 46206-6288, US

(21) Appl. No.: 10/553,163

(22) PCT Filed: Apr. 22, 2004
(86) PCT No.: PCT/US04/10960

§ 371 Date: Oct. 13, 2005

Related U.S. Application Data
(60) Provisional application No. 60/466,501, filed on Apr. 29, 2003.
(60) Provisional application No. 60/466,500, filed on Apr. 29, 2003.
(60) Provisional application No. 60/470,118, filed on May 13, 2003.

(51) Int. Cl. A61K 38/28; C07K 14/62

(52) U.S. Cl. 514/3; 530/303

(57) ABSTRACT

The present invention provides the insulin analog A0^{Arg} A21^{Gly} B31^{Arg} B32^{Arg}, which provides a protracted, even basal duration of action. The present invention also provides a method of treating diabetes mellitus comprising administering the insulin analog.

This application claims priority benefit of U.S. provisional application No. 60/466,501, filed Apr. 29, 2003, U.S. provisional application No. 60/466,500, filed Apr. 29, 2003, and U.S. provisional application No. 60/470,118, filed May 13, 2003, each of which are incorporated herein by reference.

### FIELD OF THE INVENTION

The present invention relates to insulin analogs that are useful for treating the hyperglycemia that is characteristic of diabetes mellitus.

### BACKGROUND OF THE INVENTION

The physiological demand for insulin can be separated into two phases: (a) the nutrient absorptive phase requiring a pulse of insulin to dispose of the meal-related blood glucose surge, and (b) the post-absorptive phase requiring a sustained delivery of insulin to regulate hepatic glucose output for maintaining optimal fasting blood glucose, also known as a “basal” insulin secretion.

Effective insulin therapy for people with diabetes mellitus generally involves the combined use of two types of exogenous insulin formulations: a rapid-acting, mealtime insulin provided by bolus injections, and a longer-acting insulin, administered by injection once or twice daily to control blood glucose levels between meals.

An ideal exogenous basal insulin would provide an extended and “flat” time action—that is, it would control blood glucose levels for at least 12 hours, and preferably for 24 hours, without significant risk of hypoglycemia.

Commercially used longer-acting insulin molecules do not provide an insulin effect for 24 hours. Accordingly, there remains a need for an insulin molecule that provides an insulin effect for up to 24 hours.

### SUMMARY OF THE INVENTION

The present invention provides the insulin analog A0^{Arg}A21^{Gly}B31^{Arg}B32^{Arg}-human insulin. The present invention also provides a composition comprising the insulin analog A0^{Arg}A21^{Gly}B31^{Arg}B32^{Arg}-human insulin. In a preferred embodiment, the composition is a pharmaceutical composition.

The present invention also provides the insulin analog A0^{Arg}A21^{Gly}B29^{Arg}B31^{Gly}B32^{Lys}-human insulin. The present invention also provides a composition comprising the insulin analog A0^{Arg}A21^{Gly}B29^{Arg}B31^{Arg}B32^{Lys}-human insulin. In a preferred embodiment, the composition is a pharmaceutical composition.

The present invention also provides a method of hyperglycemia, the method comprising administering to a subject an insulin analog of the present invention in an amount sufficient to regulate blood glucose concentration.

### DETAILED DESCRIPTION OF THE INVENTION

The A0^{Arg}A21^{Gly}B31^{Arg}B32^{Arg}-human insulin analog of the present invention can be visualized having the A-chain of Formula I and the B-chain of Formula II.

“Formula I” is

| | |
|---|---|
| A0 A1 A2 A3 A4 A5 A6 A7 A8 A9 A10 | (Seq. ID No. 1) |
| Arg-Gly-Ile-Val-Glu-Gln-Cys-Cys-Thr-Ser-Ile- | |
| A11 A12 A13 A14 A15 A16 A17 A18 | |
| Cys-Ser-Leu-Tyr-Gln-Leu-Glu-Asn- | |
| A19 A20 A21 | |
| Tyr-Cys-Gly. | |

The amino acids at positions A0 to A21 of Formula I correspond, respectively, to the amino acids at positions 1-22 of Seq. ID No. 1. The amino acids at positions A1 to A20 of Formula I and at positions 2-21 of Seq. ID No. 1 correspond to the amino acids at positions 1-20 of the A-chain of human insulin (Seq. ID No. 3).

“Formula II” is

| | |
|---|---|
| B1 B2 B3 B4 B5 B6 B7 B8 B9 B10 B11 | (Seq. ID No. 2) |
| Phe-Val-Asn-Gln-His-Leu-Cys-Gly-Ser-His-Leu- | |
| B12 B13 B14 B15 B16 B17 B18 B19 | |
| Val-Glu-Ala-Leu-Tyr-Leu-Val-Cys- | |
| B20 B21 B22 B23 B24 B25 B26 B27 | |
| Gly-Glu-Arg-Gly-Phe-Phe-Tyr-Thr- | |
| B28 B29 B30 B31 B32 | |
| Pro-Lys-Thr-Arg-Arg. | |

The amino acids at positions B1 to B32 of Formula II correspond, respectively, to the amino acids at positions 1-32 of Seq. ID No. 2. The amino acids at positions B1 to B30 of Formula II and at positions 1-30 of Seq. ID No. 2 correspond to the amino acids at positions 1-30 of the B-chain of human insulin (Seq. ID No. 4).

Polynucleotide and amino acid sequences of insulin molecules from a number of different species are well known to those of ordinary skill in the art. Preferably, “insulin” means human insulin. “Human insulin” has a twenty-one amino acid A-chain, which is Gly-Ile-Val-Glu-Gln-Cys-Cys-Thr-Ser-Ile-Cys-Ser-Leu-Tyr-Gln-Leu-Glu-Asn-Tyr-Cys-Asn (Seq. ID No. 3), and a thirty-amino acid B-chain, which is Phe-Val-Asn-Gln-His-Leu-Cys-Gly-Ser-His-Leu-Val-Glu-Ala-Leu-Tyr-Leu-Val-Cys-Gly-Glu-Arg-Gly-Phe-Phe-Tyr-Thr-Pro-Lys-Thr (Seq. ID No. 4).

The A- and B-chains in human insulin and of the A0^{Arg}A21^{Gly}B31^{Arg}B32^{Arg}-human insulin analog of the present invention are cross-linked by disulfide bonds. One interchain disulfide bond is between the Cys at position A7 of Formula I and the Cys at position B7 of Formula II, and the other interchain disulfide bond is between the Cys at position A20 of Formula I and the Cys at position B19 of Formula II. An intrachain disulfide bond is between the Cysteines at positions A6 and A11 of Formula L.

A simple shorthand notation is used herein to denote insulin and proinsulin molecules. For A0^{Arg}A21^{Gly}B31^{Arg}B32^{Arg}-human insulin, the shorthand notation is set forth with reference to the A-chain of Formula I (Seq. ID No. 1), the B-chain of Formula II (Seq. ID No. 2), the A-chain of human insulin (Seq. ID No. 3), and the B-chain of human insulin (Seq. ID No. 4). A0^{Arg} means that an Arg is covalently bound to the Gly at the A1 position of Seq. ID No. 3. A21^{Gly} means that the Asn in wild-type human insulin (Seq. ID No. 3) is replaced with Gly. B31^{Arg} means that an Arg is covalently bound to the Thr at the B30 position of Seq. ID No. 4. B32^{Arg} means that an Arg is covalently bound to the Arg at the B31 position of Seq. ID No. 2. Collectively, these changes to the wild-type human insulin A- and B-chains provide the insulin analog “A0^{Arg}A21^{Gly}B31^{Arg}B32^{Arg}-human insulin.” The notation “A0^{Arg}A21^{Gly}B31^{Arg}B32^{Arg}-human insulin” is synonymous with the notation “A0Arg, A21Gly, B31Arg, B32Arg-human insulin” and with the notation “ArgA0, GlyA21, ArgB31, ArgB32-human insulin.”

The A0^{Arg}A21^{Gly}B29^{Arg}B31^{Arg}B32^{Lys}-human insulin analog of the present invention can be visualized having the A-chain of Formula I and the B-chain of Formula III. As discussed above, “Formula I” is

| | |
|---|---|
| A0 A1 A2 A3 A4 A5 A6 A7 A8 A9 A10 | (Seq. ID No. 1) |
| Arg-Gly-Ile-Val-Glu-Gln-Cys-Cys-Thr-Ser-Ile- | |
| A11 A12 A13 A14 A15 A16 A17 A18 | |
| Cys-Ser-Leu-Tyr-Gln-Leu-Glu-Asn- | |
| A19 A20 A21 | |
| Tyr-Cys-Gly. | |

“Formula III” is

| | |
|---|---|
| B1 B2 B3 B4 B5 B6 B7 B8 B9 B10 B11 | (Seq. ID No. 5) |
| Phe-Val-Asn-Gln-His-Leu-Cys-Gly-Ser-His-Leu- | |
| B12 B13 B14 B15 B16 B17 B18 B19 | |
| Val-Glu-Ala-Leu Tyr-Leu-Val-Cys- | |
| B20 B21 B22 B23 B24 B25 B26 B27 | |
| Gly-Glu-Arg-Gly-Phe-Phe-Tyr-Thr- | |
| B28 B29 B30 B31 B32 | |
| Pro-Arg-Thr-Arg-Lys. | |

The amino acids at positions B1 to B32 of Formula III correspond, respectively, to the amino acids at positions 1-32 of Seq. ID No. 5. The amino acids at positions B13 to B28 and B30 of Formula III and at positions 1-28 and 30 of Seq. ID No. 5 correspond to the amino acids at positions 1-28 and 30 of the B-chain of human insulin (Seq. ID No. 4).

One interchain disulfide bond is between the Cys at position A7 of Formula I and the Cys at position B7 of Formula III, and the other interchain disulfide bond is between the Cys at position A20 of Formula I and the Cys at position B19 of Formula III. An intrachain disulfide bond is between the Cysteines at positions A6 and A11 of Formula I.

Using the simple shorthand notation discussed above, A0^{Arg} means that an Arg is covalently bound to the Gly at the A1 position of Seq. ID No. 3. A21^{Gly} means that the Asn in wild-type human insulin (Seq. ID No. 3) is replaced with Gly. B29^{Arg} means that the Lys at position B29 of the wild-type human insulin is replaced by Arg. B31^{Arg} means that an Arg is covalently bound to the Thr at the B30 position of Seq. ID No. 4. B32^{Lys} means that a Lys is covalently bound to the Arg at the B31 position of Seq. ID No. 5. Collectively, these changes to the wild-type human insulin A- and B-chains provide the insulin analog “A0^{Arg}A21^{Gly}B29^{Arg}B31^{Arg}B32^{Lys}-human insulin.” The notation “A0^{Arg}A21^{Gly}B29^{Arg}B31^{Arg}B32^{Lys}-human insulin” is synonymous with the notation “A0Arg, A21Gly, B29Arg, B31Arg, B32Lys-human insulin” and with the notation “ArgA0, GlyA21, ArgB29, ArgB31, LysB32-human insulin.”

The present invention also provides insulin analogs and proinsulin analogs in which one or more of the Arginines (Arg) is replaced with homoArginine (hArg). Thus, the present invention provides, for example, A0^{hArg}A21^{Gly}B31^{hArg}B32^{hArg}-human insulin, A0^{hArg}A21^{Gly}B29^{hArg}B31^{hArg}B32^{Lys}-human insulin, A0^{hArg}A21^{Gly}B31^{hArg}B32^{hArg}-human proinsulin, A0^{hArg}A21^{Gly}B29^{hArg}B31^{hArg}B32^{Lys}-human proinsulin.

The terms “a host cell” and “the host cell” refer to both a single host cell and to more than one host cell.

“Insulin molecule” as used herein encompasses wild-type insulins and insulin analogs.

“Proinsulin molecule” as used herein encompasses wild-type proinsulins and proinsulin analogs.

“Insulin analog” is an insulin molecule that is modified to differ from a wild-type insulin. Thus, an insulin analog can have A- and/or B-chains that have substantially the same amino acid sequences as the A-chain and the B-chain of human insulin, respectively, but differ from the A-chain and B-chain of human insulin by having one or more amino acid deletions in the A- and/or B-chains, and/or one or more amino acid replacements in the A- and/or B-chains, and/or one or more amino acids covalently bound to the N- and/or C-termini of the A- and/or B-chains.

“Proinsulin analog” is a proinsulin molecule that is modified to differ from a wild-type proinsulin. Thus, a proinsulin analog can have an A-chain, a B-chain and a C-peptide that have substantially the same amino acid sequences as the A-chain, B-chain and C-peptide in human proinsulin, respectively, but differ from the A-chain, B-chain and C-peptide of human proinsulin by having one or more amino acid deletions in the A-chain, B-chain or C-peptide, and/or one or more amino acid replacements in the A-chain, B-chain or C-peptide. In one preferred embodiment, the proinsulin analog is A21^{Gly}-human proinsulin, which, after removal of the connecting peptide, provides A0^{Arg}A21^{Gly}B31^{Arg}B32^{Arg}-human insulin. In another preferred embodiment, the proinsulin analog is A21^{Gly}B29^{Arg}B32^{Lys}-human proinsulin, which after removal of the connecting peptide, provides A0^{Arg}A21^{Gly}B29^{Arg}B31^{Arg}B32^{Lys}-human insulin. In another embodiment, the proinsulin analog A21^{Gly}B0^{Lys}B29^{Arg}B32^{Lys}-human proinsulin, which after removal of the connecting peptide, provides A0^{Arg}A21^{Gly}B29^{Arg}B31^{Arg}B32^{Lys}-human insulin. In a preferred embodiment, the proinsulin analog is purified. In another preferred embodiment, the proinsulin analog is isolated.

“Insulin template” means the insulin molecule that is chemically modified to form an insulin analog of the present invention. Insulin molecules that can be used as templates for subsequent chemical modification, include, but are not limited to, any one of the naturally occurring insulins, and preferably human insulin. Preferably, the insulin template is a recombinant insulin. More preferably, the insulin template is recombinant human insulin or an analog thereof. Most preferably the insulin template is recombinant human insulin.

“Recombinant protein” means a protein that is expressed in a eukaryotic or prokaryotic cell from an expression vector containing a polynucleotide sequence that encodes the protein. Preferably, the recombinant protein is a recombinant insulin molecule.

“Recombinant insulin molecule” is an insulin molecule that is expressed in a eukaryotic or prokaryotic cell from an expression vector that contains polynucleotide sequences that encode the A-chain and/or B-chain of an insulin molecule.

“Recombinant proinsulin molecule” is a proinsulin molecule that is expressed in a eukaryotic or prokaryotic cell from an expression vector that contains polynucleotide sequences that encode the A-chain, B-chain, and connecting peptide of a proinsulin molecule. In one preferred embodiment, the recombinant proinsulin molecule is A21^{Gly}-human proinsulin. In another preferred embodiment, the recombinant proinsulin molecule is A21^{Gly}B29^{Arg}B32^{Lys}-human proinsulin.

“Recombinant human insulin” means recombinant insulin having the wild-type (i.e., native) human A-chain (Seq. ID No. 3) and B-chain (Seq. ID No. 4) amino acid sequences.

“Sufficient to regulate blood glucose in a subject” means that administration of an insulin molecule results in a clinically normal fasting plasma glucose level. A clinically normal fasting plasma glucose level is 70-110 mg/dl. A clinically normal postprandial plasma glucose level is less than 140 mg/dl. As is well-known to those of ordinary skill in the art, insulin effect can be quantified using the “glucose clamp” technique, in which the amount of exogenous glucose required over time to maintain a predetermined plasma glucose level is used as a measure of the magnitude and duration of an insulin effect caused by an insulin molecule. For example, see Burke et al., Diabetes Research, 4:163-167 (1987). Typically, in a glucose clamp investigation, glucose is infused intravenously. If an insulin molecule causes a decrease in plasma glucose level, the glucose infusion rate is increased, such that the predetermined plasma glucose level is maintained. When the insulin molecule effect diminishes, the glucose infusion rate is decreased, such that the predetermined plasma glucose level is maintained.

“Insulin effect” means that in a glucose clamp investigation, administration of an insulin molecule requires that the rate of intravenous blood glucose administration be increased in order to maintain a predetermined plasma glucose level in the subject for the duration of the glucose clamp experiment. In one preferred embodiment, the predetermined glucose level is a normal fasting plasma glucose level. In another preferred embodiment, the predetermined glucose level is a normal postprandial plasma glucose level.

An insulin molecule has a “protracted duration of action” if the insulin molecule provides an insulin effect in hyperglycemic, e.g., diabetic, patients that lasts longer than human insulin. In a preferred embodiment, the insulin molecule provides an insulin effect for from about 8 hours to about 24 hours after a single administration of the insulin molecule. In another preferred embodiment, the insulin effect lasts from about 10 hours to about 24 hours. In another preferred embodiment, the effect lasts from about 12 hours to about 24 hours. In another preferred embodiment, the effect lasts from about 16 hours to about 24 hours. In another preferred embodiment, the effect lasts from about 20 hours to about 24 hours.

An insulin molecule has a “basal insulin effect” if the insulin molecule provides a glucose lowering effect in subjects that lasts about 24 hours after a single administration of the insulin molecule. In a preferred embodiment, the insulin molecule provides a glucose lowering effect of approximately constant magnitude (i.e., is peakless) over a period of about 24 hours after a single administration.

An “effective amount” of the insulin analog or compositions of the present invention is the quantity which results in a desired insulin effect without causing unacceptable side-effects when administered to a subject in need of insulin therapy. An “effective amount” of the insulin analog of the present invention administered to a subject will also depend on the type and severity of the disease and on the characteristics of the subject, such as general health, age, sex, body weight and tolerance to drugs. The skilled artisan will be able to determine appropriate dosages depending on these and other factors. Typically, a therapeutically effective amount of the insulin analog of the present invention can range from about 0.01 mg per day to about 1000 mg per day for an adult. Preferably, the dosage ranges from about 0.1 mg per day to about 100 mg per day, more preferably from about 1.0 mg/day to about 10 mg/day.

The dose, route of administration, and the number of administrations per day will be determined by a physician considering such factors as the therapeutic objectives, the nature and cause of the patient's disease, the patient's gender and weight, level of exercise, eating habits, the method of administration, and other factors known to the skilled physician. In broad range, a daily dose would be in the range of from about 1 nmol/kg body weight to about 6 nmol/kg body weight (6 nmol is considered equivalent to about 1 unit of insulin activity). A dose of between about 2 and about 3 nmol/kg is typical of present insulin therapy.

The present invention provides a container comprising an insulin analog of the present invention. In a preferred embodiment, the container is a vial. In another preferred embodiment, the vial contains a pharmaceutical formulation comprising an insulin analog of the present invention. In another preferred embodiment, the container is a cartridge for use in a delivery device, e.g., an insulin pen injection system. The present invention also provides an insulin pen injection system containing a cartridge containing a pharmaceutical formulation comprising an insulin analog of the present invention. The present invention also provides a unit dose system containing a pharmaceutical formulation comprising an insulin analog of the present invention.

A “desired therapeutic effect” includes one or more of the following: 1) an amelioration of the symptom(s) associated with diabetes mellitus, 2) a delay in the onset of symptoms associated with diabetes mellitus, 3) increased longevity compared with the absence of the treatment, and 4) greater quality of life compared with the absence of the treatment. For example, an “effective amount” of the insulin analog of the present invention for the treatment of diabetes is the quantity that would result in greater control of blood glucose concentration than in the absence of treatment, thereby resulting in a delay in the onset of diabetic complications such as retinopathy, neuropathy or kidney disease.

The present invention provides a method of treating hyperglycemia, the method comprising administering an insulin analog, a composition or a pharmaceutical composition of the present invention to a subject in an amount sufficient to regulate blood glucose concentration in the subject. In a preferred embodiment, the subject is treated for diabetes mellitus.

The present invention provides for the use of an insulin analog of the present invention for the manufacture of a medicament for treating hyperglycemia. The present invention also provides for the use of an insulin analog of the present invention for the manufacture of a medicament for treating diabetes mellitus.

The insulin analog of the present invention, and compositions thereof, can be administered parenterally. Parenteral administration can include, for example, systemic administration, such as by intramuscular, intravenous, subcutaneous, or intraperitoneal injection. Preferably, the route of administration is subcutaneous.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

“Isolated protein” as used herein means that the protein is removed from the environment in which it is made. A naturally occurring protein is isolated when it is removed from the cellular milieu in which the protein exists. A recombinant protein is isolated when it is removed from the cellular milieu in which the protein is expressed. A chemically modified protein, whether naturally occurring or recombinant, is isolated when it is removed from the reaction mixture in which the protein is chemically modified. Preferably, an isolated protein is removed from other proteins, polypeptides, or peptides. Methods for isolating a protein include centrifugation, chromatography, lyophilization, or electrophoresis. Such protein isolation methods and others are well known to those of ordinary skill in the art. Preferably, the insulin analog and proinsulin analog of the present invention is isolated.

Amino acids used to make an insulin analog or proinsulin analog of the present invention can be either the D- or 1-form, and can be either naturally-occurring amino acids or artificial amino acids.

In a preferred embodiment, the insulin analog of the present invention is not covalently bound to a fatty acid. In another preferred embodiment, the insulin analog of the present invention is not acylated with a fatty acid.

“Pharmaceutically acceptable” means clinically suitable for administration to a human. A pharmaceutically acceptable formulation does not contain toxic elements, undesirable contaminants or the like, and does not interfere with the activity of the active compounds therein.

“Pharmaceutical composition” means a composition that is clinically acceptable to administer to a human subject. The insulin analogs of the present invention can be formulated in a pharmaceutical composition such that the protein interacts with one or more inorganic bases, and inorganic or organic acids, to form a salt. Thus, the composition and pharmaceutical composition of the present invention can comprise a salt of an insulin analog of the present invention.

Acids commonly employed to form acid addition salts are inorganic acids such as hydrochloric acid, hydrobromic acid, hydroiodic acid, sulfuric acid, phosphoric acid, and the like, and organic acids such asp-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Preferably the pharmaceutical composition of the present invention is an acidic aqueous solution. In one preferred embodiment, the acidic aqueous solution pH is from about 3 to about 5, wherein “about” means plus or minus 0.1 pH unit. In another preferred embodiment, the pH is from about 3.5 to about 4.5. In another preferred embodiment, the pH is from about 3.75 to about 4.25. In another preferred embodiment, the acidic aqueous solution pH is about 4. Most preferably, the acidic aqueous solution pH is 4.

The insulin analog of the present invention, and compositions thereof, can be administered to the subject in conjunction with one or more pharmaceutically acceptable excipients, carriers or diluents as part of a pharmaceutical composition for treating hyperglycemia. Provided herein is a composition comprising the insulin analog of the present invention and at least one ingredient selected from the group consisting of an isotonicity agent, a divalent cation, a hexamer-stabilizing compound, a preservative, and a buffer.

Suitable pharmaceutical carriers may contain inert ingredients which do not interact with the insulin analog of the present invention. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include glycerol, lactose, dextrose, sucrose, trehalose, sorbitol, and mannitol.

“Isotonicity agent” means a compound that is physiologically tolerated and imparts a suitable tonicity to a formulation to prevent the net flow of water across cell membranes that are in contact with an administered formulation. Glycerol, which is also known as glycerin, and mannitol, are commonly used isotonicity agents. Other isotonicity agents include salts, e.g., sodium chloride, and monosaccharides, e.g., dextrose and lactose. Preferably the isotonicity agent is glycerol. The composition and pharmaceutical composition of the present invention can comprise an isotonicity agent.

“Hexamer-stabilizing compound” means a non-proteinaceous, small molecular weight compound that stabilizes the insulin analog of the present invention in a hexameric association state. Phenolic compounds, particularly phenolic preservatives, are the best known stabilizing compounds for insulin molecules. Preferably, the hexamer-stabilizing compound is one of phenol, m-cresol, o-cresol, p-cresol, chlorocresol, methylparaben, or a mixture of two or more of those compounds. More preferably, the hexamer-stabilizing compound is phenol, m-cresol, or a mixture thereof. The composition and pharmaceutical composition of the present invention can comprise a hexamer-stabilizing compound.

“Preservative” refers to a compound added to a pharmaceutical formulation to act as an anti-microbial agent. The preservative used in formulations of the present invention may be a phenolic preservative, and may be the same as, or different from the hexamer-stabilizing compound. A parenteral formulation must meet guidelines for preservative effectiveness to be a commercially viable multi-use product. Among preservatives known in the art as being effective and acceptable in parenteral formulations are benzalkonium chloride, benzethonium, chlorohexidine, phenol, m-cresol, benzyl alcohol, methylparaben, chlorobutanol, o-cresol, p-cresol, chlorocresol, phenylmercuric nitrate, thimerosal, benzoic acid, butyl paraben, ethyl paraben, phenoxy ethanol, a phenyl ethylalcohol, propyl paraben, benzylchlorocresol, chlorocresol, and various mixtures thereof. The composition and pharmaceutical composition of the present invention can comprise a preservative.

“Phenolic preservative” includes the compounds phenol, m-cresol, o-cresol, p-cresol, chlorocresol, methylparaben, and mixtures thereof. Certain phenolic preservatives, such as phenol and m-cresol, are known to bind to inn-like molecules and thereby to induce conformational changes that increase either physical or chemical stability, or both. Preferably, the phenolic preservative is m-cresol or phenol. The composition and pharmaceutical composition of the present invention can comprise a phenolic preservative.

“Buffer” or “pharmaceutically acceptable buffer” refers to a compound that is safe for use in insulin formulations and that has the effect of controlling the pH of the formulation at the pH desired for the formulation. The composition and pharmaceutical composition of the present invention can comprise a pharmaceutically acceptable buffer. Pharmaceutically acceptable buffers for controlling pH at a moderately acidic pH to a moderately basic pH include such compounds as lactate; tartrate; phosphate, and particularly sodium phosphate; acetate, and particularly sodium acetate; citrate, and particularly sodium citrate; arginine; TRIS; and histidine. “TRIS” refers to 2-amino-2-hydroxymethyl-1,3,-propanediol, and to any pharmacologically acceptable salt thereof. The free base and the hydrochloride form are two common forms of TRIS. TRIS is also known in the art as trimethylol aminomethane, tromethamine, and tris(hydroxy-methyl)aminomethane. Other pharmaceutically acceptable buffers that are suitable for controlling pH at the desired level are known to the chemist of ordinary skill.

The insulin analogs of the present invention can be taken by a subject who also takes a mealtime insulin molecule. One preferred mealtime insulin molecule is B28^{Lys}B29^{Pro}-insulin (so-called “lispro” insulin), in which the Pro at position 28 of the wild-type insulin B-chain and the Lys at position 29 of the wild-type insulin B-chain (Seq. ID No. 4) have been switched. See, for example, U.S. Pat. Nos. 5,514,646, 5,474,978 and 5,700,662. Another mealtime insulin molecule is B28^{Asp}-insulin, in which the wild-type Pro at position 28 of the B-chain has been replaced by Asp. See U.S. Pat. No. 5,547,930. Another mealtime insulin molecule is B31^{Lys}B29^{Glu}-insulin. See U.S. Pat. No. 6,221,633.

The insulin analog of the present invention can be complexed with a suitable divalent metal cation. “Divalent metal cation” means the ion or ions that participate to form a complex with a multiplicity of protein molecules. The transition metals, the alkaline metals, and the alkaline earth metals are examples of metals that are known to form complexes with insulin. The transition metals are preferred. Preferably, the divalent metal cation is one or more of the cations selected from the group consisting of zinc, copper, cobalt, manganese, calcium, cadmium, nickel, and iron. More preferably, zinc is the divalent metal cation. Preferably, zinc is provided as a salt, such as zinc sulfate, zinc chloride, zinc oxide, or zinc acetate.

To obtain the complexes between the insulin analog of the present invention and a divalent metal cation, the protein is dissolved in a suitable buffer and in the presence of a metal salt. The mixture is allowed to incubate at ambient temperature to allow the complex to precipitate. Suitable buffers are those which maintain the mixture at a pH range from about 3.0 to about 9.0 and do not interfere with the complexation reaction. Examples include phosphate buffers, acetate buffers, citrate buffers and Goode's buffers, e.g., HEPES, Tris and Tris acetate. Suitable metal salts are those in which the metal is available for complexation. Examples of suitable zinc salts include zinc chloride, zinc acetate, zinc oxide, and zinc sulfate.

An insulin analog of the present invention can be obtained using recombinant methodologies. For example, a recombinant proinsulin analog can be used. Alternatively, recombinant insulin A- and B-chains can be expressed in host cells and then recombined. Alternatively, an insulin precursor can be used. Each of these methodologies are well known to those of ordinary skill in the art. For example, see U.S. Pat. No. 4,421,685, U.S. Pat. No. 4,569,791, U.S. Pat. No. 4,569,792, U.S. Pat. No. 4,581,165, U.S. Pat. No. 4,654,324, U.S. Pat. No. 5,304,473, U.S. Pat. No. 5,457,066, U.S. Pat. No. 5,559,094 European patent application EP 741188 A1. See also Chance et al., Diabetes Care 16 (Suppl 3): 133-142 (1993); Chance et al., “Peptides: Synthesis-Structure-Function,” in: Proceedings of the 7^{th} American Peptide Symposium, Rich, D. H. et al., eds., Pierce Chemical Company, Rockford, Ill., pp. 721-738 (1981); and Frank et al., Munch med Wsch 125 (Suppl. 1): S14-20 (1983).

A proinsulin analog can be enzymatically processed to form the insulin analog by cleaving with carboxypeptidase B and trypsin. Alternatively, the proinsulin analog can be cleaved using lysyl endoproteinase, e.g., LysC endoproteinase, and trypsin. The reaction conditions are adjusted such that the lysyl endoproteinase does not cleave at the B29Lys.

The medium used to culture the cells may be any conventional medium suitable for growing the host cells, such as minimal or complex media containing appropriate supplements. Suitable media are available from commercial suppliers or may be prepared of published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of peptide in question.

The present invention also provides a nucleic acid comprising a nucleic acid sequence encoding a proinsulin analog of the present invention. In a preferred embodiment, the proinsulin analog is A21^{Gly}-human proinsulin. In another preferred embodiment, the proinsulin analog is A21^{Gly}B29^{Arg}B32^{Lys}-human proinsulin.

The present invention also provides a vector comprising a nucleic acid sequence encoding a proinsulin analog of the present invention. The present invention also provides a host cell comprising the vector. In a preferred embodiment, the host cell is a eukaryotic cell. Preferably, the eukaryotic cell is a fungal cell, a yeast cell, a mammalian cell, or an immortalized mammalian cell line cell. More preferably, the eukaryotic cell is a yeast cell. In another preferred embodiment, the host cell is a prokaryotic cell. Preferably, the prokaryotic cell is a bacterial cell, and more preferably is an E. coli cell.

The present invention also provides a composition comprising a proinsulin analog of the present invention. In a preferred embodiment, cell culture medium comprising a proinsulin analog of the present invention is provided.

Also provided is a method of expressing a proinsulin analog of the present invention, comprising cultivating a host cell containing a nucleic acid sequence encoding a prosinsulin analog of the present invention under conditions suitable for propagation of the host cell and for expression of the proinsulin analog. In one preferred embodiment, the method further comprises purifying the proinsulin analog from the host cell. In another preferred embodiment, the method further comprises purifying the proinsulin analog from the culture medium. In yet another preferred embodiment, the method further comprises purifying the proinsulin analog from both the host cell and from the culture media.

Also provided is a recombinant method of expressing an analog of the present invention, comprising (a) cultivating a host cell containing a nucleic acid sequence encoding a prosinsulin analog having a connecting peptide sequence under conditions suitable for propagation of the host cell and for expression of the proinsulin analog, (b) purifying the proinsulin analog, and removing the connecting peptide. In a preferred embodiment, the connecting peptide is removed using one or more enzymes, for example, trypsin and/or carboxypeptidase B. In another preferred embodiment, a leader is present in the proinsulin analog, and the leader is removed.

The present invention also provides a nucleic acid comprising a nucleic acid sequence encoding the A- and/or B-chains of an insulin analog of the present invention. In a preferred embodiment, the insulin analog is A0^{Arg}A21^{Gly}B31^{Arg}B32^{Arg}-human insulin. In another preferred embodiment, the insulin analog is A0^{Arg}A21^{Gly}B29^{Arg}B31^{Arg}B32^{Lys}-human insulin. After expression and purification, the A- and B-chains of the insulin analog are combined to form an insulin analog of the present invention.

The present invention also provides a vector comprising a nucleic acid sequence encoding the A- and/or B-chains of an insulin analog of the present invention. The present invention also provides a host cell comprising the vector. In a preferred embodiment, the host cell is a eukaryotic cell. Preferably, the eukaryotic cell is a fungal cell, a yeast cell, a mammalian cell, or an immortalized mammalian cell line cell. More preferably, the eukaryotic cell is a yeast cell. In another preferred embodiment, the host cell is a prokaryotic cell. Preferably, the prokaryotic cell is a bacterial cell, and more preferably is an E. coli cell.

Also provided is a method of expressing an insulin analog of the present invention, comprising cultivating a host cell containing a nucleic acid sequence encoding the A- and/or B-chain of an insulin analog of the present invention under conditions suitable for propagation of the host cell and for expression of the A- and/or B-chains. In one preferred embodiment, the method further comprises purifying the A- and/or B-chains from the host cell. In another preferred embodiment, the method further comprises purifying the A- and/or B-chains from the culture medium. In yet another preferred embodiment, the method further comprises purifying the A- and/or B-chains from both the host cell and from the culture medium.

Nucleic acid sequence encoding the proinsulin analog or A- and/or B-chains may be inserted into any vector which may conveniently be subjected to recombinant DNA procedures, and the choice of vector will often depend on the host cell into which it is to be introduced. Thus, the vector may be an autonomously replicating vector, i.e., a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the peptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide of the invention in a variety of host cells are well known in the art.

The DNA sequence encoding the peptide may also, if necessary, be operably connected to a suitable terminator, polyadenylation signals, transcriptional enhancer sequences, and translational enchancer sequences. The recombinant vector of the invention may further comprise a DNA sequence enabling the vector to replicate in the host cell in question.

The vector may also comprise a selectable marker, e.g., a gene the product of which complements a defect in the host cell or one which confers resistance to a drug, e.g. ampicillin, kanamycin, tetracyclin, chloramphenicol, neomycin, hygromycin or methotrexate.

To direct a parent peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The present invention also provides a cell culture medium comprising a proinsulin analog of the present invention. The present invention also provides a cell culture medium comprising the A- and/or B-chains of an insulin analog of the present invention. In a preferred embodiment, the medium also contains host cell material. In another preferred embodiment, the medium is substantially free of host cell material.

The insulin analog of the present invention can also be prepared by using standard methods of solid-phase peptide synthesis techniques. Peptide synthesizers are commercially available from, for example, Applied Biosystems in Foster City Calif. Reagents for solid phase synthesis are commercially available, for example, from Midwest Biotech (Fishers, Ind.). In one embodiment, an automated solid phase peptide synthesizer can be used according to the manufacturer's instructions for blocking interfering groups, protecting the amino acid to be reacted, coupling, decoupling, and capping of unreacted amino acids. In another embodiment, an automated solid phase peptide synthesizer can be used according to the manufacturer's instructions for peptide synthesis via successive cylcles of amino acid coupling, capping of unreacted alpha-amino groups, and deprotection. Peptide synthesis can also be performed manually with similar procedures.

Peptides can be synthesized using standard automated solid-phase synthesis protocols using t-butoxycarbonyl- or fluorenylmethoxycarbonyl-alpha-amino acids with appropriate side-chain protection. After completion of synthesis, peptides are cleaved from the solid-phase support with simultaneous side-chain deprotection using standard hydrogen fluoride or TFA methods. Crude peptides are then purified, for example, using Reversed-Phase Chromatography on Vydac C18 columns using linear water-acetonitrile gradients in which all solvents contain 0.1% TFA. To remove acetonitrile and water, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

### EXAMPLE 1

### Acylation of A21^{Gly}B31^{Arg}B32^{Arg}-Human Insulin with Boc-Arg(Pbf)-NHS Ester to Produce A0^{Arg}A21^{Gly}B31^{Arg}B32^{Arg}-Human Insulin

Boc-Arg(Pbf)-NHS ester (0.4 mmol) is prepared from 0.4 mmol each of Boc-Arg(Pbf)-OH, N-hydroxysuccinimide (NHS), and dicyclohexylcarbodiimide (DCC) mixed together in dichloromethane for 30 min. The mixture is then filtered, concentrated to dryness on a rotary evaporator, and dissolved in 4 mL of MeOH. 180 mg of A21^{Gly}B31^{Arg}B32^{Arg}-human insulin (0.030 mmol) is dissolved in 20 mL of 50:50 water/CH₃CN containing 50 mM Na₂HPO₄, and the pH is adjusted 7.7 with 5 M KOH solution. 1.2 mL of the Boc-Arg(Pbf)-NHS ester solution (0.12 mmol) is added to the insulin solution. The reaction is stirred at room temperature for 50 min, then an additional 1.2 mL of Boc-Arg(Pbf)-NHS ester solution (0.12 mmol) is added, and it is allowed to react for an additional 30 min, followed by acidification with 100 μL TFA. The sample is analyzed by reversed-phase HPLC with mass spectral detection (LC-MS) on a Zorbax Eclipse XDB-C8 4.6 mm i.d.×15 cm column with a linear AB gradient of 10 to 100% B over 15 min in which A=0.05% TFA/H₂O and B=0.05% TFA in 60:40 CH₃CN:H₂O and the flow rate is 1 mL/min. The mixture contains some remaining underivatized protein, three monoacylated peaks in relative ratios of approximately 7.5:1.5:1 and three diacylated peaks in relative ratios of 2:5:3.

Based on previous experiments, the major monoacylated product is expected to be the product resulting from acylation at the N terminus of the A chain, and this is the compound which is isolated and characterized. The reaction mixture is purified on a Kromasil C₁₈ 1.2 cm i.d.×25 cm preparative column (10 μm particle size) at 8 mL/min. The sample is eluted using a two-stage linear AB gradient of 0 to 20% B over 15 min followed by 20 to 70% B over 100 min, where A=0.05% TFA/H₂O and B=0.05% TFA/CH₃CN. The combined purified material is lyophilized to give 28 mg of the major monoderivatized compound. The lyophilized protein is dissolved in 20 mL of 94:2:2:2 TFA:anisole:triisopropylsilane:MeOH and left at room temperature for 1.5 hr. The sample is then evaporated to <2 mL on a rotory evaporator, diluted with 25 mL of 10:90 acetonitrile:water and extracted three times with ethyl ether. A final purification is performed as above but with the two-stage linear AB gradient of 0 to 15% B over 15 min followed by 15 to 55% B over 100 min. The combined pure fractions are lyophilized and yielded 21 mg (approximately 12% yield). The observed molecular weight is 6218.3 (calculated molecular weight=6219.1). The identity of the product is confirmed by digestion of 0.1 mg of the protein with 5 μg of Endoproteinase Glu-C (from Staphylococcus aureus V8; Sigma P6181) in pH 8 buffer for 2 hr at 37 degrees. LC-MS analysis of the resultant mixture indicated the presence of the diagnostic fragments A(1-4)+Arg; A(1-17)-B(1-13)+Arg; and A(5-17)-B(1-13), thus confirming the location of the added Arg residue at the N terminus of the A chain.

### EXAMPLE 2

### In Vitro Receptor Affinity

The affinity of insulin analogs for the human insulin receptor (IR) is measured in a competitive binding assay using radiolabeled ligand, [¹²⁵I] insulin. Human insulin receptor membranes are prepared as P1 membrane preparation of stable transfected 293EBNA cells overexpressing the receptor. The assay is developed and validated in both filtration and SPA (scintillation proximity assay) mode with comparable results, but is performed in the SAP mode employing PVT PEI treated wheatgerm agglutinin-coupled SPA beads, Type A (WGA PVT PEI SPA) beads from Amersham Pharmacia Biotech.

Radiolabeled ligand ([¹²⁵I] recombinant human insulin) is prepared in house or purchased from Amersham Pharmacia Biotech, at specific activity 2000 Ci/mmol on the reference date. SPA assay buffer is 50 mM Tris-HCL, pH 7.8, 150 mM NaCl, 0.1% BSA. The assay is configured for high throughput in 96-well microplates (Costar, # 3632) and automated with radioligand, membranes and SPA beads added by Titertec/Plus (ICN Pharmaceuticals).

The reagents are added to the plate wells in the following order:

| Reagent | Final concentration |
|---|---|
| Insulin analog or control human | Min signal (BHI) = 0.1 μM, all other |
| insulin dilution | compounds [Hi] = 0.1 μM |
| [¹²⁵I] recombinant human insulin | 50 pM |
| HIR membranes | 1.25 μg |
| WGA PVT PEI SPA beads | 0.25 mg/well |

The plates are sealed with an adhesive plate cover and shaken for 1 min on LabLine Instruments tier plate shaker. The plates are incubated at room temperature (22⁰ C) for 12 hours by placing them in a Wallac Microbeta scintillation counter and setting the timer for 12 hours. The counting is done for 1 min per well using protocol normalized for [¹²⁵I]. IC₅₀ for each insulin molecule is determined from 4-parameter logistic non-linear regression analysis. Data is reported as mean±SEM. Relative affinity is determined by comparing an insulin analog to the recombinant human insulin control within each experiment and then averaging the relative affinity over the number of experiments performed. Therefore, a comparison of the average IC₅₀ for an insulin analog with the average IC₅₀ for insulin does not generate the same value.

The affinity of an insulin analog and recombinant human insulin for insulin growth factor receptor (IGF 1-R) is measured in the competitive binding assay using [¹²⁵I]IGF-1 radiolabeled ligand. Human IGF-1 receptor membranes are prepared as P1 membrane preparation of stable transfected 293EBNA cells overexpressing the receptor. The assay is developed and validated in both filtration and SPA (scintillation proximity assay) mode with comparable results, but is routinely performed in the SAP mode employing PVT PEI treated wheatgerm agglutinin-coupled SPA beads, Type A (WGA PVT PEI SPA) beads from Amersham Pharmacia Biotech. [¹²⁵I]IGF-1 radiolabeled ligand is prepared in house or purchased from Amersham Pharmacia Biotech, at specific activity 2000 Ci/mmol on the reference date. SPA assay buffer is 50 mM Tris-HCL, pH 7.8, 150 mM NaCl, 0.1% BSA. The assay is configured for high throughput in 96-well microplates (Costar, #3632) and automated with radioligand, membranes and SPA beads added by Titertec/Plus (ICN Pharmaceuticals).

The reagents are added to the plate wells in the following order.

| Reagent | Final concentration |
|---|---|
| Insulin analog or control human | Min signal (IGF-1) = 1 μM, all other |
| insulin dilution | compounds [Hi] = 10 μM |
| [¹²⁵I] IGF-1 | 50 pM |
| IGF-1R membranes | 1.25 μg |
| WGA PVT PEI SPA beads | 0.25 mg/well |

The plates are sealed with adhesive plate cover and shaken for 1 min on LabLine Instruments tier plate shaker. The plates are incubated at room temperature (22° C.) for 12 hours by placing them in a Wallac Microbeta scintillation counter and setting the timer for 12 hours. The counting is done for 1 min per well using protocol normalized for [¹²⁵I].

IC₅₀ for each insulin molecule is determined from 4-parameter logistic non-linear regression analysis. Data is reported as mean±SEM. Relative affinity is determined by comparing an insulin analog to the recombinant insulin control within each experiment and then averaging the relative affinity over the number of experiments performed. Therefore, a comparison of the average IC₅₀ for an insulin analog with the average IC₅₀ for insulin does not generate the same value.

The selectivity index is calculated as the ratio of IR relative affinity to IGF-1 R relative affinity. A selectivity index>1 indicates a greater relative selectivity for IR. A selectivity index<1 indicates a greater relative selectivity for IGF-R.

Table 1 depicts insulin receptor (IR) affinity, insulin-like growth factor 1 (IGF1-R) receptor affinity, and a receptor selectivity index (IR/IGF1-R) for the insulin analog s and and recombinant human insulin.

TABLE 1

| | Relative | | | Relative | | | |
|---|---|---|---|---|---|---|---|
| | IR Affinity | | | IGF1-R Affinity | | | |
| Molecule | Mean | SEM | n | Mean | SEM | n | Index |
| recombinant human insulin | 1.00 | 0.00 | 86 | 1.00 | 0.00 | 85 | 1.00 |
| A0^{Arg}A21^{Gly}B31^{Arg}B32^{Arg}-human insulin | 0.32 | 0.03 | 3 | 1.2 | 0.12 | 3 | 0.26 |
| A0^{Arg}A21^{Gly}B29^{Arg}B31^{Arg}B32^{Lys}-human insulin | 0.21 | 0.01 | 3 | 1.1 | 0.21 | 3 | 0.20 |

### EXAMPLE 3

### In Vitro Metabolic Potency

Metabolic potency (glucose uptake) of A0^{Arg}A21^{Gly}B31^{Arg}B32^{Arg}-human insulin and recombinant human insulin is determined in the glucose-uptake assay using differentiated mouse 3T3-L1 adipocytes. Undifferentiated mouse 3T3 cells are plated at density 25,000 cells/well in 100 μl of growth media (DMEM, high glucose, w/out L-glutamine, 10% calf serum, 2 mM L-glutamine, 1% antibiotic/antimycotic solution).

Differentiation is initiated 3 days after plating by addition of differentiation media: DMEM, high-glucose, w/out L glutamine, 10% FBS, 2 mM L-Glutamine, 1% antibiotic/antimycotic solution, 10 mM HEPES, 0.25 mM dexamethasone, 0.5 mM 3-isobutyl-1-methylxanthine (IBMX), 5 mg/ml insulin. After 48 hours (day 3), the differentiation media is changed to one with insulin, but without IBMX or dexamethasone and at day 6 the cells are switched to differentiation media containing no insulin, IBMX or dexamethasone. The cells are maintained in FBS media, with changes every other day.

Glucose transport assay is performed using Cytostar T 96 well plates. 24 hours prior to assay cells were switched to 100 μl of serum free media containing 0.1% of BSA. On the day of the assay, the media is removed and 50 μl of assay buffer is added: a so-called KRBH or Krebs-Ringer buffer containing HEPES, pH 7.4 (118 mM NaCL, 4.8 mM KCl, 1.2 mM MgSO₄×7H₂0, 1.3 mM CaCl₂H₂0, 1.2 mM KH₂PO₄, 15 mM HEPES). Insulin dilutions are prepared in same buffer with 0.1% BSA, and added as 2×. The blank contains KRBH, 0.1% BSA and 20 mM 2×2-deoxy-D-Glucose, 0.2 μCi/well of 2-deoxy-D-(U-¹⁴C) glucose and 2×10⁻⁷ insulin. The cells are incubated at 37⁰ C for 1 hour. After that period 10 μl of cytochalasin B is added to a final concentration of 200 μM in KRBH, and the plates are read on a Microbeta plate reader. Relative affinity is determined by comparing A0^{Arg}A21^{Gly}B31^{Arg}B32^{Arg}-human insulin to the recombinant human insulin control within each experiment and then averaging the relative affinity over the number of experiments performed. Therefore, a comparison of the average EC₅₀ for A0^{Arg}A21^{Gly}B31^{Arg}B32^{Arg}-human insulin to the average EC₅₀ for insulin does not generate the same value.

Table 2 depicts the in vitro metabolic potency for A0^{Arg}A21^{Gly}B31^{Arg}B32^{Arg}-human insulin and recombinant human insulin.

TABLE 2

Metabolic Potency

| Molecule | Mean | N |
|---|---|---|
| recombinant human insulin | 1.00 | >50 |
| A0^{Arg}A21^{Gly}B31^{Arg}B32^{Arg}-human insulin | 0.23 | 2 |

### EXAMPLE 4

### In Vitro Mitogenicity

The mitogenic potency of A0^{Arg}A21^{Gly}B31^{Arg}B32^{Arg}-human insulin is determined by measuring proliferation of human mammary epithelial cells (HMEC) in culture. HMEC are obtained from Clonetics Corporation (San Diego, Calif.) at passage 7 and are expanded and frozen at passage 8. A fresh ampoule is used for each time so that all experiments are conducted with the same passage 10 of HMEC. Cells are maintained in culture according to Bio Whittaker instructions. To maintain the cell culture, the growth medium is changed every other day and the cultures are inspected daily.

Two products from BioWhittaker are used as the growth medium:

1. Fully supplemented MEGM (CC-3051), including: (amounts indicate final concentrations, except BPE)

10 ng/ml hEGF (human recombinant Epidermal Growth Factor)

5 μg/ml Insulin

0.5 μg/ml Hydrocortisone

50 μg/ml Gentamicin, 50 ng/ml Amphotericin-B

13 mg/ml BPE (Bovine pituitary Extract) 2 ml (attached); and

2. Basal Medium (MEBM, CC-3151) with all the supplements listed below (SingleQuots, CC-3150)

13 mg/ml BPE (Bovine Pituitary Extract (CC-4009) 2 ml

10 μg/ml hEGF (CC-4017) 0.5 ml

5 μg/ml Insulin (CC-4031) 0.5 ml

0.5 mg/ml Hydrocortisone (CC-4031) 0.5 ml

50 mg/ml Gentamicin, 50 mg/ml Amphotericin-B (CC-4081) 0.5 ml.

For a growth experiment, the assay medium is growth medium without 5 μg/l Insulin, and with 0.1% BSA. The assay is performed in 96 well Cytostart scintillating microplates (Amersham Pharmacia Biotech, RPNQ0162). Recombinant human insulin and IGF-1 are controls used in each assay run, and recombinant human insulin is on each assay plate.

The assays are performed according to the following protocol. On day one, HMECs are seeded at a density of 4000 cells/well in 100 μl of Assay Medium. Insulin in the growth medium is replaced with graded doses of recombinant human insulin or an other insulin molecule from 0 to 1000 nM final concentration. After 4-hour incubation, 0.1 μCi of ¹⁴C-thymidine in 10 μl of assay medium is added to each well and plates are read at 48 h and/or 72 h on Trilux.

Typically, the maximal growth response is between 3-4-fold stimulation over basal. Response data are normalized to between 0 and 100% response equal to 100× (response at concentration X−response at concentration zero) divided by (response at maximal concentration−response at zero concentration). Concentration-response data are fit by non-linear regression employing JMP software.

Relative mitogenic potency is determined by comparing A0^{Arg}A21^{Gly}B31^{Arg}B32^{Arg}-human insulin to insulin control within each experiment and then averaging the relative potency over the number of experiments performed. Therefore, a comparison of the average EC₅₀ for A0^{Arg}A21^{Gly}B31^{Arg}B32^{Arg}-human insulin with the average EC₅₀ for insulin does not generate the same value.

Table 3 depicts the in vitro mitogenicity, measured in terms of cell proliferation, for A0^{Arg}A21^{Gly}B31^{Arg}B32^{Arg}-human insulin and recombinant human insulin. The data in Table 3 show that A0^{Arg}A21^{Gly}B31^{Arg}B32^{Arg}-human insulin is less mitogenic than recombinant human insulin.

TABLE 3

| | Mitogenic Potency | | |
|---|---|---|---|
| Molecule | Mean | SEM | N |
| recombinant human insulin | 1.00 | 0.00 | 250 |
| A0^{Arg}A21^{Gly}B31^{Arg}B32^{Arg}-human insulin | 0.86 | 0.05 | 4 |

### EXAMPLE 5

### Phosphate Buffered Saline Solubility

An in vitro precipitation assay that is indicative of a propensity to extend time-action in vivo is developed as follows. An aqueous solution adjusted to pH 4 and containing a pharmacological dose (100 international units) of an insulin molecule and 30 μg/ml of Zn²⁺, 2.7 mg/ml of m-cresol and 17 mg/ml glycerol % is neutralized with phosphate buffered saline (PBS) to 2 international units and centrifuged for 5 min at 14,000 rpm and RT. The supernatant is removed and approximately one tenth of the supernatant is injected into an analytical Symmetry Shield RP8 RP-HPLC system (Waters, Inc.). Area under the eluted peak is integrated and compared to area under the peak of reference standard, which is either recombinant human insulin in 0.1N HCl. The ratio of the areas is multiplied by 100 to generate % solubility in PBS.

The PBS solubility for the recombinant human insulin formulation and for the insulin analogs is shown in Table 4.

TABLE 4

| Molecule | PBS Solubility |
|---|---|
| recombinant human insulin | 89.5 |
| A0^{Arg}A21^{Gly}B31^{Arg}B32^{Arg}-human insulin | 9.6 |
| A0^{Arg}A21^{Gly}B29^{Arg}B31^{Arg}B32^{Lys}-human insulin | 26.8 |

### EXAMPLE 6

### Isoelectric Point

Isoelectric focusing is an electrophoretic technique that separates proteins on the basis of their isoelectric points (pI). The pI is the pH at which a protein has no net charge and does not move in an electric field. IEF gels effectively create a pH gradient so proteins separate on their unique pI property. Detection of protein bands can be accomplished by sensitive dye staining like Novex Collodial Coomassie Staining Kit. Alternatively, detection can be achieved by blotting the gel onto polyvinylidene difluoride (PVDF) membrane and staining it with Ponceau Red. The pI of a protein is determined by comparing it to pI of a known standard. IEF protein standards are combination of proteins with well-characterized pI values blended to give uniform staining. Yet another method of pI determination is IEF by capillary electrohoresis (cIEF). The pI is determined by comparison to known markers.

The isoelectric point (pI) of recombinant human insulin and A0^{Arg}A21^{Gly}B31^{Arg}B32^{Arg}-human insulin is determined by isoelectric focusing gel electrophoresis using Novex IEF gels of pH 3-10 that offer pI performance range of 3.5-8.5. The isoelectric points are shown in Table 5.

TABLE 5

| Molecule | Isoelectric Point |
|---|---|
| recombinant human insulin | 5.62 |
| A0^{Arg}A21^{Gly}B31^{Arg}B32^{Arg}-human insulin | 7.30 |

### EXAMPLE 7

### Biosynthesis of A0^{Arg}A21^{Gly}B29^{Arg}B31^{Arg}B32^{Lys}-Human Insulin

A recombinant plasmid containing the sequence encoding A21^{Gly}B0^{Lys}B29^{Arg}B32^{Lys}-human proinsulin is used to transform a commercially available E. coli expression strain in order to produce the proinsulin molecule. The leader sequence began with an initiator Met codon, and is followed by a Lys residue to form the B0^{Lys} in the proinsulin molecule. The overexpressed protein accumulates in inclusion bodies, which are collected after final spin of the cell lysate at 4,000 rpm. Leader-A21^{Gly}B0^{Lys}B29^{Arg}B32^{Lys}-human proinsulin protein is solubilized in 50 mM Tris, pH 9.0, 7M urea, and the protein mixture is then put through a pH ramp to pH 11.0 for 1 hour, at room temperature with stirring to further enhance the solubilization. Sulfitolysis with sodium sulfite (100 mM final concentration) and sodium tetrathionate (4.9 mM final concentration) proceeds for 4 hours at room temperature with gentle stirring. The sulfitolized protein is then purified by Pharmacia Fast Flow Q Sepharose chromatography run in 50 mM Tris, pH 9.0, 7M Urea with a 0 to 400 mM NaCl gradient elution. The purified protein solution is then diluted to approximately 150 μg/ml with 20 mM Glycine, pH 10.5. Folding is initiated by addition of Cysteine to 2 mM final concentration and carried out for 60-72 hours at 4⁰ C with gentle stirring. The folded protein is purified using Pharmacia Fast Flow SP Sepharose chromatography run in 100 mM NaAcetate, pH 4.0, 30% acetonitrile with a linear NaCl gradient elution. The pooled fractions containing purified protein are lyophilized, redissolved in water and loaded on a C18 reversed phase column and eluted with an acetonitrile gradient. The collected eluent containing the proinsulin is lyophilized to yield 210 mg protein.

Cleavage on the C-terminal side of the three Lysine residues at B0, B32, and C64 is expected to lead to the direct generation of the desired insulin analog. 22 mg of proinsulin (MW 9990.4; 0.0022 mmol) is dissolved in 2 mL of 25 mM tris, pH 8.2 buffer, and 25 μg of endoproteinase Lys-C (Sigma catalog number P-3428; obtained from Lysobacter enzymogenes) is added to the protein solution which is incubated at 370 for 5 hr then allowed to sit at room temperature for an additional 15 hr. At this point, there is a significant amount of white precipitate in the sample. It is centrifuged at 3000 rpm for 5 min and the supernatant is removed. The precipitate is redissolved in 3 mL of 30:70 acetonitrile:water at pH 2.

An analytical reversed phase HPLC trace of the supernatant reveals a peak at 10.77 min (major component of the supernatant), which has a molecular weight of 3148.5 Da, which is exactly the expected molecular weight for the C-peptide fragment obtained from cleavage at B32^{Lys}/B33^{Glu} and C64^{Lys}/C65^{Arg}.

An analytical reversed phase HPLC trace of the precipitate reveals a peak at 11.25 min (major component of the precipitate), which has a molecular weight of 6219.8 Da which compares very well with the calculated molecular weight for the desired product (6219.1 Da). Therefore, the product of interest is obtained in the precipitate. It appears that a small percentage of the product of interest is also left in the supernatant.

The product is repurified on a Vydac C18 reversed phase HPLC column (10 mm i.d.×25 cm) with a flow rate of 6 mL/min and a linear 0.5% acetonitrile/min gradient.

The lyophilized final product yields 9.0 mg for a recovery efficiency from the proinsulin form of approximately 66% (100% efficiency would give 62% of the starting mass due to the loss of the leader and C peptide mass).

All patents, patent applications, articles, books, and other publications cited herein are incorporated by reference in their entireties.

1. A pharmaceutical composition comprising an acidic aqueous solution comprising the insulin analog A0^{Arg}A21^{Gly}B31^{Arg}B32^{Arg}-human insulin, wherein the insulin analog is not covalently bound to a fatty acid.

2. (canceled)

3. (canceled)

4. The pharmaceutical composition of claim 1, further comprising one or more pharmaceutically acceptable excipients.

5. The pharmaceutical composition of claim 1, further comprising a divalent metal cation.

6. The pharmaceutical composition of claim 5, wherein the divalent metal cation is zinc.

7. The pharmaceutical composition of claim 1, further comprising a phenolic preservative.

8. The pharmaceutical composition of claim 1, further comprising an isotonicity agent.

9. A method of treating hyperglycemia, the method comprising administering the pharmaceutical composition of claim 1 to a subject in an amount sufficient to regulate blood glucose concentration in the subject.

10. A method of treating hyperglycemia, the method comprising administering the pharmaceutical composition of any one of claims 4, 5, 6, 7, 8 or 9 to a subject in an amount sufficient to regulate blood glucose concentration in the subject.

11. The method of claim 9, wherein the subject is treated for diabetes mellitus.

12. (canceled)

13. (canceled)

14. (canceled)

15. (canceled)

16. (canceled)

17. (canceled)

18. (canceled)

19. (canceled)

20. (canceled)

21. (canceled)

22. The method of claim 10, wherein the subject is treated for diabetes mellitus.


SEQUENCE LISTING (5 sequences)

SEQ ID NO: 1 - protein, 22 aa, HOMO SAPIENS
  MISC_FEATURE (1)..(22): Amino acid sequence of the A-chain of A0ArgA21GlyB31ArgB32Arg-human insulin and A0ArgA21GlyB29ArgB31Arg B32Lys-human insulin.
RGIVEQCCTSICSLYQLENYCG

SEQ ID NO: 2 - protein, 32 aa, homo sapiens
  MISC_FEATURE (1)..(32): Amino acid sequence of the B-chain of A0ArgA21GlyB31ArgB32Arg-human insulin.
FVNQHLCGSHLVEALYLVCGERGFFYTPKTRR

SEQ ID NO: 3 - protein, 21 aa, homo sapiens
  MISC_FEATURE (1)..(21): Amino acid sequence of the A-chain of wild-type human insulin.
GIVEQCCTSICSLYQLENYCN

SEQ ID NO: 4 - protein, 30 aa, homo sapiens
  MISC_FEATURE (1)..(30): Amino acid sequence of the B-chain of wild-type human insulin.
FVNQHLCGSHLVEALYLVCGERGFFYTPKT

SEQ ID NO: 5 - protein, 32 aa, homo sapiens
  MISC_FEATURE (1)..(32): Amino acid sequence of the B-chain of A0ArgA21GlyB29ArgB31ArgB32Lys -human insulin.
FVNQHLCGSHLVEALYLVCGERGFFYTPRTRK